(19) United States
(12) Patent Application Publication
(10) Pub. No.: US 2023/0103300 A1
(43) Pub. Date: Apr. 6, 2023

(54) TOPICAL PHARMACEUTICAL COMPOSITION IN GEL FORM COMPRISING AT LEAST AMITRIPTYLINE FOR USE IN THE TREATMENT OF CORONAVIRUS-INDUCED NEUROPATHIC PAIN

(71) Applicant: AlgoTherapeutix, Suresnes (FR)

(72) Inventors: Nicolas Paola Principe, Cernay La Ville (FR); Frédéric Lallemand, Rambouillet (FR); Céline Greco, Paris (FR); Stéphane Thiroloix, Suresnes (FR)

(73) Assignee: AlgoTherapeutix, Suresnes (FR)

(21) Appl. No.: 17/936,813

(22) Filed: Sep. 29, 2022

(30) Foreign Application Priority Data
Oct. 1, 2021 (FR) 2110425

(51) Int. Cl. A61K 31/137; A61K 9/00; A61K 9/06; A61K 47/38; A61K 47/10

(52) U.S. Cl. CPC A61K 31/137; A61K 9/0014; A61K 9/06; A61K 47/38; A61K 47/10

(57) ABSTRACT

The present invention relates to a method for treating coronavirus-induced neuropathic pain comprising the topical application of a pharmaceutical composition containing amitriptyline or one of the pharmaceutically acceptable salts thereof.

### CROSS-REFERENCE TO RELATED APPLICATION

This application claims the benefit of French Application No. 2110425, filed Oct. 1, 2021, the disclosure of which is hereby incorporated by reference in its entirety.

The present invention relates to a method for treating coronavirus-induced neuropathic pain comprising the topical application of a pharmaceutical composition containing amitriptyline or one of the pharmaceutically acceptable salts thereof.

Peripheral neuropathic pain is caused by damage to nerve structures such as peripheral nerve endings or nociceptors which become extremely sensitive to stimulation and which can generate pulsing in the absence of stimulation.

This damage can be caused by many reasons such as injuries, illnesses such as diabetes, shingles and advanced-stage cancers, a chemical burn or chemotherapy treatments. More specifically, the inherent toxicity of chemotherapy treatments directly harms the axon and can give rise to nerve demyelination.

The application WO2018/197307 (filed by Algotherapeutix, and also published as US 2020/0197326) and the article by Rossignol, J. et al. (“High concentration of topical amitriptyline for treating chemotherapy-induced neuropathies”. Support Care Cancer 27, 3053-3059 (2019)), describe the use of topical compositions in the form of creams with a high amitriptyline concentration for the treatment of post-chemotherapy peripheral neuropathic pain.

Recently, several scientific publications, for example:

Mao et al. “Neurologic manifestations of hospitalized patients with coronavirus disease 2019 in Wuhan, China”. JAMA Neurology. 2020 Apr. 10;
Abdelnour L. et al. “COVID-19 infection presenting as a motor peripheral neuropathy”. Journal of the Formosan Medical Association (2020) 119, 1119-1120;
Alberti P. et al. “Guillain-Barré syndrome related to COVID-19 infection”. Neurol Neuroimmunol Neuroinflamm. 2020;7: e741;
mentioned the existence of a correlation between SARS-CoV-2 (the coronavirus causing COVID-19) infection and the onset of neurological sequelae, particularly peripheral, or even Guillain-Barré syndrome (rare disorder in which the patient's immune system attacks the peripheral nerves).

The scientific article by Dalakas M. C. (“Guillain-Barré syndrome: The first documented COVID-19—triggered autoimmune neurologic disease” Neurol Neuroimmunol Neuroinflamm. 2020;7:e781) even suggests that SARS-CoV-2 may be the cause of several types of autoimmune neurological diseases.

However, to our knowledge, no known medication or therapies are available to date for the specific treatment of neuropathic pain induced by a coronavirus such as SARS-CoV-2.

Therefore, there is a real need to develop a pharmaceutical composition which is effective in the treatment of neuropathic pain, particularly peripheral, induced by a coronavirus type virus, such as for example SARS-CoV-2.

It was discovered surprisingly that topically applied amitriptyline-based compositions made it possible to effectively treat neuropathic pain induced by a coronavirus type virus, such as for example SARS-CoV-2.

This discovery is all the more surprising given that coronavirus-induced neuropathic pain and chemotherapy-induced neuropathic pain do not appear to have the same cause.

Indeed, as stated above, chemotherapy-induced pain is primarily due to the intrinsic chemical toxicity of the drugs used in chemotherapy, which will directly harm the axons and may give rise to nerve demyelination.

Whereas coronavirus-induced neuropathic pain seems to be the result of an immune reaction of the body to the infection.

However, the compositions in cream form described in the application WO2018/197307 and in the article by Rossignol, J. et al, are not entirely satisfactory in terms of stability over time.

Indeed, creams are the dosage forms routinely used for administering active substances topically, as they generally provide a superior transdermal passage and good solubilization of all the agents. However, the physicochemical stability of the composition according to the application WO2018/197307 in cream form, and more specifically in oil-in-water emulsion form, is not satisfactory, particularly for use thereof as a medicinal product.

Amitriptyline hydrochloride is an amphiphilic amphiphile which is water-soluble in salt form. However, it was discovered unexpectedly that the presence of these electrolytes is destabilizing for oil-in-water emulsions, apparently by masking oil globule surface charging and by disrupting oil/water interface equilibria. This destabilization induces a phase separation of the emulsion and ultimately total separation of the oil and water. At the same time, a chemical reaction occurs, conveyed by a yellowing of the initially white color of the emulsion. This phase separation and this yellowing are problematic for potential marketing of the composition, particularly as a medicinal product.

Therefore, there is a real need to develop a pharmaceutical composition which is effective in the treatment of neuropathic pain, particularly peripheral, induced by a coronavirus type virus, such as for example SARS-CoV-2, and which is particularly stable over time and to temperature.

More particularly, it is of interest that the pharmaceutical composition has a good physicochemical stability even at a high amitriptyline concentration (i.e., even at a concentration of at least 10% by weight of amitriptyline with respect to the total weight of the composition).

It is also of interest that the composition has good qualities of use.

However, it is was surprisingly discovered that a topical pharmaceutical composition in aqueous gel form comprising at least amitriptyline and/or one of the pharmaceutically acceptable salts thereof, with a total content of amitriptyline and/or one of the pharmaceutically acceptable salts thereof between 10 and 30% by weight with respect to the total weight of the composition, makes it possible to effectively treat neuropathic pain induced by a coronavirus type virus, such as for example SARS-CoV-2, and has a good physicochemical stability over time.

Therefore, the invention relates to a method for treating coronavirus-induced neuropathic pain comprising the topical application of a pharmaceutical composition in aqueous gel form containing from 10 to 30% by weight, with respect to the total weight of the composition, of amitriptyline and/or one of the pharmaceutically acceptable salts thereof.

It was observed that the composition according to the invention is particularly effective for the treatment of neuropathic pain induced by a coronavirus type virus, such as SARS-CoV-2 (Severe Acute Respiratory Syndrome CoronaVirus 2), MERS-CoV (Middle East Respiratory Syndrome CoronaVirus) or SARS-CoV-1 (Severe Acute Respiratory Syndrome CoronaVirus 1).

More particularly, the composition according to the invention is highly effective for the treatment of neuropathic pain, particularly peripheral, induced by the SARS-CoV-2 virus.

Similarly, the composition according to the invention is highly effective for the treatment of neuropathic pain, particularly peripheral, in people suffering from COVID-19.

It was also observed that the pharmaceutical composition according to the invention helps facilitate the penetration of amitriptyline through the skin with low systemic passage, and thus obtain good therapeutic efficacy.

The composition according to the invention moreover has an enhanced bioavailability, preferably at concentrations of amitriptyline and/or of one of the pharmaceutically acceptable salts thereof of 10 to 25% by weight and in particular between 10 and 20% by weight with respect to the total weight of the composition. Indeed, at a high concentration, amitriptyline tends to be reorganized, inducing the formation of aggregates capable of limiting bioavailability.

Furthermore, the compositions according to the invention are particularly stable over time at ambient temperature, but also at higher storage temperatures. The compositions according to the invention underwent stability studies under conditions of ambient temperature (25° C.) for at least 24 months and accelerated temperature (40° C.) for 6 months. Following from these studies, the compositions according to the invention did not change visual appearance either physically (viscosity) or chemically (pH, assay of active substance and degradation products).

A composition according to the invention also underwent forced degradation under conditions of high acidity, high alkalinity, heat, light and oxidative conditions. The degradation products observed remained within acceptable limits of the standard ICH Q3B (International Council for Harmonisation).

Furthermore, the compositions according to the invention comprise few excipients, which promotes good local tolerance of the composition (lower risk of allergy, lower risk of irritation). The topical application of the composition according to the invention has little or no side-effects.

The compositions according to the invention also have good properties for use, that is to say, the compositions are translucent, odorless and pleasant to touch (non-greasy texture).

In addition, the compositions according to the invention are administered very easily with a pump bottle. These pump bottles are particularly useful for ensuring good reproducibility and good precision of the administered dose of active substance.

Furthermore, the composition according to the invention could additionally remedy pain, make it possible to restore healthier and more hydrated skin.

Further features, aspects and advantages of the invention will emerge more clearly on reading the following description and examples.

In the present description, and unless specified otherwise:
the expression “at least one” is equivalent to the expression “one or more” and is interchangeable therewith;
the expression “between . . . and . . . ” is equivalent to the expression “ranging from . . . to . . . ” and is interchangeable therewith, and implies that the bounds are included;
the expression “fatty alcohol” denotes an alcohol comprising from 9 to 40 carbon atoms;
the expression “fatty acid” denotes an acid comprising from 9 to 40 carbon atoms;
the expression “fatty ether” denotes an ether comprising from 9 to 40 carbon atoms;
the expression “fatty ester” denotes an ester comprising from 9 to 40 carbon atoms;
the expression “polyoxyalkylenated” corresponds, according to the invention, to a unit—(O-alkyl)ₙ—, where n is an integer varying from 2 to 200, preferably from 2 to 40, more preferably from 2 to 20, and where alkyl preferably represents an ethyl or a propyl;
the expression “polyoxyethylenated” corresponds, according to the invention, to a unit—(O—CH₂CH₂)n —, where n is an integer varying from 2 to 200, preferably from 2 to 40, more preferably from 2 to 20.

The pharmaceutical composition according to the invention is used in the topical treatment of neuropathic pain induced by a coronavirus type virus, such as the SARS-CoV-2 virus.

Advantageously, the pharmaceutical composition according to the invention is used in the topical treatment of peripheral neuropathic pain induced by a coronavirus type virus, such as the SARS-CoV-2 virus.

Very specifically, the pharmaceutical composition according to the invention is used in the topical treatment of peripheral neuropathic pain induced by SARS-CoV-2. Similarly, the pharmaceutical composition according to the invention is used in the topical treatment of neuropathic pain, particularly peripheral, induced by COVID-19.

Advantageously, the pharmaceutical composition according to the invention is preferably used by the cutaneous route. In other words, the pharmaceutical composition is preferably applied on the skin, particularly of the hands and feet.

Advantageously, the pharmaceutical composition can equally well be used for preventive purposes (for people infected with coronavirus not yet experiencing neuropathic pain) and/or for curative purposes (for people infected with coronavirus already experiencing neuropathic pain).

Amitriptyline

The composition according to the present invention comprises at least amitriptyline and/or one of the pharmaceutically acceptable salts thereof.

According to the invention, the total content of amitriptyline and/or of one of the pharmaceutically acceptable salts thereof is between 10 and 30% by weight, with respect to the total weight of the composition.

Amitriptyline has the following formula (I):

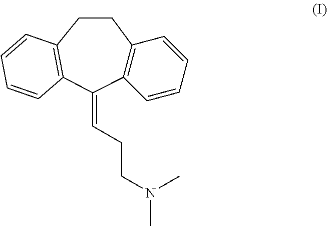 [SMILES: CN(C)CCC=C1c2ccccc2CCc2ccccc21]

Within the scope of the present invention, the term “pharmaceutically acceptable amitriptyline salts” denotes salts compatible with a pharmaceutical composition, i.e., intended to be administered to humans. In particular, the term pharmaceutically acceptable amitriptyline salts denotes hydrates, solvates, acid salts such as hydrochlorides and clathrates of amitriptyline.

As a very particularly preferred amitriptyline salt, amitriptyline hydrochloride will be used.

Preferably, the total content of amitriptyline and/or of one of the pharmaceutically acceptable salts thereof is between 10 and 25% by weight, more preferably between 10 and 20% by weight, even more preferably between 10 and 15% by weight, with respect to the total weight of the composition.

More preferably, the total content of amitriptyline hydrochloride is between 10 and 25% by weight, more preferably between 10 and 20% by weight, even more preferably between 10 and 15% by weight, with respect to the total weight of the composition.

Very specifically, it was surprisingly observed that, when the total content of amitriptyline, and/or of one of the pharmaceutically acceptable salts thereof such as amitriptyline hydrochloride, is between 10 and 25% by weight, more preferably between 10 and 20% by weight, with respect to the total weight of the composition according to the invention, then the bioavailability of amitriptyline of the composition according to the invention is significantly enhanced.

Indeed, it was observed that, at a high concentration, amitriptyline tends to be reorganized, inducing the formation of aggregates capable of limiting bioavailability.

Preferably, the composition according to the invention contains amitriptyline and/or one of the pharmaceutically acceptable salts thereof in the proportions cited above, as the sole agent treating pain.

The pharmaceutical composition according to the invention is in aqueous gel form.

According to the Clinical Data Interchange Standards Consortium (CDISC), a pharmaceutical gel is a semi-solid dosage form containing a gelling agent to give a solution or a colloidal dispersion rigidity. A gel can contain suspended particles.

According to the invention, it is understood that the composition in aqueous gel form according to the invention comprises viscous aqueous compositions in which the viscosity is between 400 and 2500 mPa·s (at a temperature of 20° C. and at atmospheric pressure).

Preferably, the viscosity of the compositions in aqueous gel form according to the invention, at a temperature of 20° C. and at atmospheric pressure, is between 400 and 2500 mPa·s; more preferably between 600 and 2000 mPa·s; and even more preferably between 800 and 1500 mPa·s.

By way of example, the viscosity of the compositions in aqueous gel form according to the invention is determined by means of a Brookfield LV viscometer, using spindle number 63, rotating at a speed of 50 rpm (revolutions per minute), at a temperature 20.0° C.+/−2.0° C.) in a 30 mL receptacle, 40 mm in height and 35 mm in diameter. When the viscometer is calibrated, the spindle is submerged in the gel up to one centimeter from the bottom of the bottle. The viscosity is read when the measurement is stable.

The composition according to the invention is not in emulsion form, such as for example an oil-in-water emulsion or a water-in-oil emulsion. In other words, the composition according to the invention comprises no oily phase.

Advantageously, the composition according to the invention is free from fatty substances.

According to the invention, the term “fatty substance” denotes an organic compound insoluble in water at 25° C. and at atmospheric pressure (760 mm Hg, or 1.013.10⁵ Pa), i.e., having a solubility in water less than 5% and preferably less than 1%, even more preferably less than 0.1%. By way of examples of fatty substances, mention can be made of waxes, hydrocarbons, fatty alcohols comprising from 9 to 40 carbon atoms, fatty acids comprising from 9 to 40 carbon atoms, fatty esters comprising from 9 to 40 carbon atoms, fatty ethers preferably comprising from 9 to 40 carbon atoms, silicones and mixtures thereof.

The composition according to the invention comprises water.

Preferably, the total water content is greater than or equal to 65% by weight, more preferably between 65 and 90% by weight; even more preferably between 70 and 90% by weight, even more preferably between 75 and 85% by weight, with respect to the total weight of the composition according to the invention.

Preferably, the composition according to the invention comprises at least one cellulose polymer.

The term “cellulose” polymer denotes according to the invention any polysaccharide compound, optionally substituted, having in the chain structure thereof glucose residues joined by 13-1,4 bonds; besides non-substituted celluloses, the cellulose derivatives can be anionic, cationic, amphoteric or non-ionic.

Thus, the cellulose polymers that can be used according to the invention can be chosen from non-substituted celluloses including in a microcrystalline form and substituted celluloses.

More preferably, the cellulose polymers than can be used contain no C₁₀-C₃₀ fatty side chain in the structure thereof.

Preferably, the cellulose polymer(s) that can be used have a mean molecular weight between 5000 and 1,500,000, more preferably between 50,000 and 800,000, even more preferably between 400,000 and 800,000.

Of the cellulose polymers that can be used according to the invention, cellulose ethers, cellulose esters and cellulose ether esters can be differentiated.

Cellulose esters include inorganic cellulose esters (cellulose nitrates, sulfates or phosphates, etc.), organic cellulose esters (cellulose monoacetates, triacetates, amidopropionates, acetatebutyrates, acetatepropionates or acetatetrimellitates, etc.) and mixed organic/inorganic cellulose esters such as cellulose acetatebutyratesulfates and acetatepropionatesulfates. Of cellulose ether esters, mention can be made of hydroxypropylmethylcellulose phthalates and ethylcellulose sulfates.

Of non-ionic cellulose ethers, mention can be made of (C₁-C₄)alkylcelluloses such as methylcelluloses and ethylcelluloses (for example Ethocel standard 100 Premium from DOW CHEMICAL); (poly)hydroxy(C₁-C₄)alkylcelluloses such as hydroxymethylcelluloses, hydroxyethylcelluloses (for example Natrosol 250 HHR sold by AQUALON) and hydroxypropylcelluloses (for example Klucel EF from AQUALON); mixed (poly)hydroxy(C₁-C₄)alkyl-(C₁-C₄)alkylcellulose celluloses such as hydroxypropyl-methylcelluloses (for example Methocel E4M from DOW CHEMICAL), hydroxyethyl-methylcelluloses, hydroxyethyl-ethylcelluloses (for example Bermocoll E 481 FQ from AKZO NOBEL) and hydroxybutyl-methylcelluloses.

Of anionic cellulose ethers, mention can be made of (poly)carboxy(C₁-C₄)alkylcelluloses and salts thereof. By way of example, mention can be made of carboxymethylcelluloses, carboxymethylmethylcelluloses (for example Blanose 7M from AQUALON) and carboxymethylhydroxyethylcelluloses and sodium salts thereof.

Of cationic cellulose ethers, mention can be made of cationic cellulose derivatives such as cellulose copolymers and cellulose derivatives grafted with a water-soluble quaternary ammonium monomer, and particularly described in the patent U.S. Pat. No. 4,131,576, such as (poly)hydroxy(C₁-C₄)alkyl celluloses, such as hydroxymethyl-, hydroxyethyl- or hydroxypropyl celluloses grafter in particular with a methacryloylethyl-trimethylammonium, methacrylmidopropyl-trimethyl ammonium, dimethyl-diallylammonium salt. The commercial products fitting this definition are more specifically the products sold under the trade name “Celquat® L 200” and “Celquat® H 100” by National Starch.

Preferably, the cellulose polymer(s) are chosen from cellulose polymers including no C₁₀-C₃₀ fatty side-chain in the structure thereof; more preferably from cellulose ethers; even more preferably from non-ionic cellulose ethers; even more preferably from (a) (C₁-C₄)alkylcelluloses such as methylcelluloses and ethylcelluloses, (b) (poly)hydroxy(C₁-C₄)alkylcelluloses such as hydroxymethylcelluloses, hydroxyethylcelluloses and hydroxypropylcelluloses, (c) mixed (poly)hydroxy(C₁-C₄)alkyl-(C₁-C₄)alkylcellulose celluloses such as hydroxypropyl-methylcelluloses, hydroxypropyl-ethylcelluloses, hydroxyethyl-methylcelluloses, hydroxyethyl-ethylcelluloses and hydroxybutyl-methylcelluloses, and (d) mixtures thereof.

More preferably, the composition according to the invention comprises at least one (poly)hydroxy(C₁-C₄)alkylcellulose such as hydroxymethylcelluloses, hydroxyethylcelluloses and hydroxypropylcelluloses; more preferably at least hydroxyethyl cellulose.

Preferably, when the composition according to the invention comprises at least one cellulose polymer, the total content of cellulose polymer(s) is between 0.1 and 10% by weight, more preferably between 0.5 and 5% by weight, even more preferably between 1 and 2.5% by weight, with respect to the total weight of the composition according to the invention.

Preferably, when the composition according to the invention comprises at least one cellulose polymer, the total content of (poly)hydroxy(C₁-C₄)alkylcellulose(s) is between 0.1 and 10% by weight, more preferably between 0.5 and 5% by weight, even more preferably between 1 and 2.5% by weight, with respect to the total weight of the composition according to the invention.

Preferably, when the composition according to the invention comprises at least one cellulose polymer, the total content of hydroxyethylcellulose is between 0.1 and 10% by weight, more preferably between 0.5 and 5% by weight, even more preferably between 1 and 2.5% by weight, with respect to the total weight of the composition according to the invention.

Preferably, the composition according to the invention comprises at least one C₂-C₈ polyol.

The term “C₂-C₈ polyol” according to the present invention denotes an organic compound consisting of a C₂-C₈ hydrocarbon chain, optionally interrupted by one or more oxygen atoms, and carrying at least two free hydroxyl groups (—OH) carried by different carbon atoms, this compound optionally being cyclic or acyclic, linear or branched, and in the liquid state at ambient temperature (25° C.) and at atmospheric pressure (i.e., 1.013.10⁵ Pa).

Preferably, the C₂-C₈ polyol(s) that can be used are acyclic and non-aromatic.

The C₂-C₈ polyols that can be used comprise in the structure thereof from 2 to 8 carbon atoms, preferably from 2 to 6 carbon atoms, more preferably from 2 to 5 carbon atoms.

More specifically, the polyol(s) that can be used comprise from 2 to 10 hydroxy groups, more preferably from 2 to 5 hydroxy groups, more preferably from 2 to 3 hydroxy groups.

Preferably, the or said C₂-C₈ polyol(s) that can be used are chosen from C₃-C₆ polyols, ethylene glycol, and mixtures thereof.

More preferably, the or said C₂-C₈ polyol(s) that can be used according to the invention are chosen from propylene glycol, 1,3-propanediol, 1,3-butylene glycol, pentane-1,2-diol, dipropylene glycol, hexylene glycol, pentylene glycol, glycerol, ethylene glycol, and a mixture of these compounds; more preferably, the composition comprises at least propylene glycol.

Preferably, when the composition according to the invention comprises at least one C₂-C₈ polyol, the total content of C₂-C₈ polyol(s) is between 0.1 and 15% by weight, more preferably between 0.5 and 10% by weight, even more preferably between 1 and 6% by weight, and even more preferably between 3 and 6% by weight, with respect to the total weight of the composition according to the invention.

Preferably, when the composition according to the invention comprises at least one C₂-C₈ polyol, the total content of propylene glycol is between 0.1 and 15% by weight, more preferably between 0.5 and 10% by weight, even more preferably between 1 and 6% by weight, and even more preferably between 3 and 6% by weight, with respect to the total weight of the composition according to the invention.

Preferably, when the composition according to the invention comprises at least one cellulose polymer and at least one C₂-C₈ polyol, the weight ratio of the total content of one C₂-C₈ polyol(s), on one hand, over the total content of cellulose polymer(s), on the other, ranges from 0.01 to 150, more preferably from 0.1 to 20, even more preferably from 0.4 to 6, more preferably from 1 to 6, or from 1.2 to 6.

Advantageously, when the composition according to the invention comprises at least one cellulose polymer and at least one C₂-C₈ polyol, the total content by weight of cellulose polymer(s) is strictly less than the total content by weight of C₂-C₈ polyol(s).

Preferably, the composition according to the invention is free from surfactant.

According to a specific embodiment of the invention, the composition can optionally further comprise at least one surfactant.

The surfactants that can be used according to the invention can be chosen from anionic surfactants, cationic surfactants, amphoteric and/or zwitterionic surfactants, non-ionic surfactants, and mixtures thereof.

More preferably, the surfactant(s) that can be used according to the invention are chosen from non-ionic surfactants.

The non-ionic surfactants that can be used can be chosen from alkyl polyglucosides (APG), oxyalkylenated glycerol esters, optionally oxyalkylenated fatty acid and sorbitan esters, polyoxyalkylenated (particularly polyoxyethylenated and/or polyoxypropylenated) fatty acid esters optionally associated with a fatty acid and glycerol ester such as the mixture PEG-100 Stearate/Glyceryl Stearate marketed for example by ICI under the trade name Arlacel 165, oxyalkylenated sugar esters, and mixtures thereof. By way of alkylpolyglucosides, mention can be made of those containing an alkyl group including from 6 to 30 carbon atoms and preferably from 8 to 16 carbon atoms, and containing a glucoside group preferably comprising 1.2 to 3 glucoside units. The alkylpolyglucosi des can be chosen for example from decylglucoside (Alkyl-C₉/C₁₁-polyglucoside (1.4)) such as the product marketed under the trade name Mydol 10® by Kao Chemicals or the product marketed under the trade name Plantacare 2000 UP® by Cognis; caprylyl/capryl glucoside such as the product marketed under the trade name Plantacare KE 3711® by Cognis; laurylglucoside such as the product marketed under the trade name Plantacare 1200 UP® by Cognis; cocoglucoside such as the product marketed under the trade name Plantacare 818 UP® by Cognis; caprylylglucoside such as the product marketed under the trade name Plantacare 810 UP® by Cognis; and mixtures thereof.

The oxyalkylenated glycerol esters are particularly polyoxyethylenated derivatives of glyceryl and fatty acid esters and the hydrogenated derivatives thereof. These oxyalkylenated glycerol esters can be chosen for example from hydrogenated and oxyethylenated glyceryl and fatty acid esters such as PEG-200 hydrogenated glyceryl palmate marketed under the trade name Rewoderm LI-S 80 by Goldschmidt; oxyethylenated glyceryl cocoates such as PEG-7 glyceryl cocoate marketed under the trade name Tegosoft GC by Goldschmidt, and PEG-30 glyceryl cocoate marketed under the trade name Rewoderm LI-63 by Goldschmidt; oxyethylenated glyceryl stearates; and mixtures thereof.

The oxyalkylenated sugar esters are particularly polyethylene glycol ethers of fatty acid and sugar esters. These oxyalkylenated sugar esters can be chosen for example from oxyethylenated glucose esters such as PEG-120 methyl glucose dioleate marketed under the trade name Glucamate DOE 120 by Amerchol.

Preferably, the number of moles of alkylene oxide of the non-ionic surfactants that can be used according to the invention varies from 2 to 400; more preferably from 4 to 250.

According to an alternative embodiment of the invention, the composition comprises at least one non-ionic surfactant; more preferably a non-ionic surfactant chosen from polyoxyalkylenated glycerol esters; even more preferably at least one non-ionic surfactant chosen from hydrogenated and polyoxyethylenated glyceryl and fatty acid esters such as PEG-200 hydrogenated glyceryl palmate, polyoxyethylenated glyceryl cocoates such as PEG-7 glyceryl cocoate and PEG-30 glyceryl cocoate, polyoxyethylenated glyceryl stearates, and mixtures thereof. Even more preferably according to this alternative embodiment, the composition comprises at least one polyoxyethylenated glyceryl cocoate.

Preferably, when the composition according to the invention comprises at least one surfactant, the total content of surfactant(s) is between 0.1 and 10% by weight, more preferably between 0.5 and 5% by weight, even more preferably between 1 and 4% by weight, with respect to the total weight of the composition according to the invention.

Preferably, when the composition according to the invention comprises at least one surfactant, the total content of non-ionic surfactant(s) is between 0.1 and 10% by weight, more preferably between 0.5 and 5% by weight, even more preferably between 1 and 4% by weight, with respect to the total weight of the composition according to the invention.

Preferably, when the composition according to the invention comprises at least one surfactant, the total content of (poly)oxyalkylenated glycerol ester(s) is between 0.1 and 10% by weight, more preferably between 0.5 and 5% by weight, even more preferably between 1 and 4% by weight, with respect to the total weight of the composition according to the invention.

Preferably, the composition according to the invention is free from antioxidant agent.

According to an alternative embodiment of the invention, the composition further comprises at least one antioxidant agent; more preferably chosen from tocopherol and esters thereof, such as tocopherol acetate, propyl gallate, butylated hydroxytoluene (BHT), butylated hydroxyanisole (BHA), and mixtures thereof.

Preferably, the composition according to the invention is free from sequestering agent.

According to an alternative embodiment of the invention, the composition further comprises at least one sequestering agent; more preferably chosen from (a) ethylenediamine tetraacetic acid (EDTA) and salts thereof such as disodium ethylene diaminetetraacetic salt (Disodium EDTA), (b) phosphonic derivatives and salts thereof such as hexamethylene diaminetetra(methylene phosphonic) acid, ethylenediamine tetra(methylene phosphonic) acid, 1-hydroxyethylidene-1,1-diphosphonic acid, aminotri(methylenephosphonic) acid, diethylene-triamine penta(methylene phosphonic) acid, (c) polyamine polymers such as polyalkylene polyamines and derivatives thereof, in particular polyethyleneimine, (d) dendrimers with chelating activity, (e) proteins such as spermine, spermidine, transferrin, ferritin, (0 carboxylic acids such as phytic acid, citric acid, malic acid, nitrilo-acetic acid, fumaric acid, tartaric acid, succinic acid, oxalic acid, (g) desferrioxamine mesylate, and mixtures thereof.

The definition of “sequestering agent” (also known as “chelating agent”) is well-known to a person skilled in the art and refers to a compound or a mixture of compounds capable of forming a chelate with a metal ion. A chelate is an inorganic complex wherein a compound (the sequestering or chelating agent) is coordinated with a metal ion, i.e., it forms one or more bonds with the metal ion (formation of a ring including the metal ion).

The sequestering (or chelating) agent generally comprises at least two electron donor atoms which enable the formation of bonds with the metal ion.

According to a further alternative embodiment of the invention, the composition comprises at least one sequestering agent and at least one antioxidant agent.

According to a further alternative embodiment of the invention, the composition is free from fatty substances, sequestering agent and/or antioxidant agent.

Preferably, the pH of the composition according to the invention is between 3 and 8, more preferably between 4 and 7, and more preferably between 5 and 6.

The pH of these compositions can be adjusted to the desired value by means of commonly used alkalinizing agents and/or acidifying agents. Of the alkalinizing agents, mention can be made, by way of examples, of ammonia, alkanolamines, mineral or organic hydroxides. Of the acidifying agents, mention can be made, by way of examples, of mineral or organic acids such as hydrochloric acid, orthophosphoric acid, carboxylic acids such as for example acetic acid, tartaric acid, citric acid, lactic acid, sulfonic acids.

The composition according to the invention can furthermore contain additives or excipients commonly used in pharmaceuticals, such as one or more fragrances, buffers, dyestuffs, antibacterials and/or antifungals.

By way of antibacterial, parabens are preferably used, and more preferably methyl-paraben.

These additives or excipients can be present in the composition according to the invention in a quantity ranging from 0 to 20% with respect to the total weight of the composition.

A person skilled in the art will take care to choose these optional additives or excipients and the quantities thereof in such a way that they do not harm the properties of the compositions according to the present invention.

Particularly preferably, the pharmaceutical composition in aqueous gel form for topical application according to the invention is the following composition CP1, comprising:
from 10 to 30% by weight, preferably from 10 to 25% by weight, more preferably from 10 to 20% by weight, even more preferably from 10 to 15% by weight, of amitriptyline or one of the pharmaceutically acceptable salts thereof, with respect to the total weight of the composition,
from 0.1 to 10% by weight, preferably from 0.5 to 5% by weight, more preferably from 1 to 2.5% by weight of at least one cellulose polymer as described above, with respect to the total weight of the composition,
from 0.1 to 15% by weight, preferably from 0.5 to 10% by weight, more preferably from 1 to 6% by weight, and even more preferably from 3 to 6% by weight, of at least one C₂-C₈ polyol as described above, with respect to the total weight of the composition,
optionally from 0.1 to 10% by weight, preferably from 0.5 to 5% by weight, more preferably from 1 to 4% by weight, of at least one non-ionic surfactant as described above, with respect to the total weight of the composition,
optionally from 0 to 3% by weight of at least one sequestering agent and/or at least one antioxidant agent as described above,
optionally from 0.01 to 0.5% by weight of at least one antibacterial agent, preferably methyl-paraben,
optionally from 0 to 1% by weight of one or more pH adjusters as described above, so as to keep the pH between 3 and 8, preferably between 4 and 7, more preferably between 5 and 6.
water, in a total content greater than or equal to 65% by weight, preferably between 65 and 90% by weight; more preferably between 70 and 90% by weight, even more preferably between 75 and 85% by weight, with respect to the total weight of the composition.

According to an alternative embodiment of this composition CP1, the composition CP1 is free from fatty substances, surfactant, sequestering agent and/or antioxidant agent.

Especially preferably, the pharmaceutical composition in aqueous gel form for topical application according to the invention is the following composition CP2, comprising:
from 10 to 30% by weight, preferably from 10 to 25% by weight, more preferably from 10 to 20% by weight, even more preferably from 10 to 15% by weight, of amitriptyline or one of the pharmaceutically acceptable salts thereof, with respect to the total weight of the composition,
from 0.1 to 10% by weight, preferably from 0.5 to 5% by weight, more preferably from 1 to 2.5% by weight, of at least one non-ionic cellulose ether, preferably with a mean molecular weight between 50,000 and 800,000, as described above, with respect to the total weight of the composition,
from 0.1 to 15% by weight, preferably from 0.5 to 10% by weight, even more preferably from 1 to 6% by weight, and more preferably from 3 to 6% by weight, of at least one C₂-C₈ polyol chosen from propylene glycol, 1,3-propanediol, 1,3-butylene glycol, pentane-1,2-diol, dipropylene glycol, hexylene glycol, pentylene glycol, glycerol, ethylene glycol, and a mixture of these compounds, with respect to the total weight of the composition,
optionally from 0.1 to 10% by weight, preferably from 0.5 to 5% by weight, more preferably from 1 to 4% by weight, of at least one oxyalkylenated glycerol ester as described above, with respect to the total weight of the composition,
optionally from 0 to 3% by weight of at least one sequestering agent and/or at least one antioxidant agent as described above,
optionally from 0.01 to 0.5% by weight of at least one antibacterial agent, preferably methyl-paraben,
optionally from 0 to 1% by weight of one or more pH adjusters as described above, so as to keep the pH between 3 and 8, preferably between 4 and 7, more preferably between 5 and 6.
water, in a total content greater than or equal to 65% by weight, preferably between 65 and 90% by weight; more preferably between 70 and 90% by weight, even more preferably between 75 and 85% by weight, with respect to the total weight of the composition.

According to an alternative embodiment of this composition CP2, the composition CP2 is free from fatty substances, surfactant, sequestering agent and/or antioxidant agent.

The compositions CP1 and CP2 are particularly effective in the treatment of SARS-CoV-2-induced peripheral neuropathic pain.

The invention also relates to a method for the treatment by the topical route, preferably by the cutaneous route, of neuropathic pain, particularly peripheral, induced by a coronavirus, preferably SARS-CoV-2, comprising one or more topical applications of the composition according to the invention as described above.

Advantageously, the pharmaceutical composition can equally well be applied for preventive purposes (for people infected with coronavirus not yet experiencing neuropathic pain) and/or for curative purposes (for people infected with coronavirus already experiencing neuropathic pain).

The following examples illustrate the compositions according to the invention and the advantages of these compositions. They are in no way a restriction of the present invention.

### EXAMPLES

### Example 1

Comparative ex vivo study of the percutaneous absorption of amitriptyline in a formulation A in aqueous gel form (invention) and in a formulation B in cream form (comparative).

The following aqueous gel (composition A) was prepared from the ingredients indicated in the table hereinafter, the quantities of which are expressed as a % by weight.

TABLE 1

| COMPOSITION A (Invention) | Quantity |
|---|---|
| Amitriptyline hydrochloride | 10 |
| Hydroxyethylcellulose | 1 |
| Propylene glycol | 5 |
| PEG-7 Glyceryl cocoate | 2 |
| Disodium ethylene diaminetetraacetic salt | 0.1 |
| Propyl gallate | 0.05 |
| pH agent | Q.s. pH 5.5 ± 0.5 |
| Water | Q.s. 100 |

Composition B (cream) comprises 10% by weight of amitriptyline hydrochloride and 90% by weight of Excipial Hydrocreme® cream, marketed by Galderma, with respect to the total weight of composition B.

Each of compositions A and B was applied on separate human skin samples. For each composition, the experiment was repeated 3 times with 3 skin samples from 3 different donors, i.e., 9 samples.

The skin samples are mounted in a Frantz cell and are heated to a surface temperature of 32° C.±1° C.

Composition A or B is spread evenly using a spatula on each skin sample at a rate of 10 mg per cell, corresponding to 5 mg/cm² of skin.

The skin samples are rinsed 16 hours after application.

Each skin sample was placed with a tweezers on a paper towel (dermis facing downward).

The stratum corneum is removed using adhesive strips.

After removing the stratum corneum, the sample is perforated. The epidermis is then separated from the dermis. Each of them is placed in separate bottles.

The different samples were then extracted.

This penetration profile demonstrated its clinical efficacy in the study described by the article by Rossignol et al (“High concentration of topical amitriptyline for treating chemotherapy-induced neuropathies”. Support Care Cancer 27, 3053-3059(2019)). The results of these extractions are compiled in the table below.

TABLE 2

| | Composition A | Composition B |
|---|---|---|
| | (Invention) | (Comparative) |
| Amitriptyline concentration remaining | 2.4 ± 1.5 | 3.3 ± 1.6 |
| on skin surface - stratum corneum (μg) | | |
| Amitriptyline concentration in | 3.6 ± 2.6 | 4.1 ± 2.5 |
| epidermis (μg) | | |
| Amitriptyline concentration in dermis | 5.2 ± 2.5 | 4.4 ± 2.2 |
| (μg) | | |
| Amitriptyline concentration in recipient | 0.15 ± 0.08 | 0.13 ± 0.13 |
| fluid (bloodstream) | | |
| Bioavailability (μg/cm² of skin) | 9.0 ± 4.8 | 8.7 ± 4.0 |

It was also observed that the systemic passage of amitriptyline was less than 0.1% of the administered dose. As a result, the systemic passage of amitriptyline is negligeable.

It is noted that the aqueous gel A according to the invention has a satisfactory skin penetration profile of amitriptyline, similar to the skin penetration profile of amitriptyline obtained with the comparative cream B.

It is also noted that the bioavailability obtained from composition A and that obtained from composition B are similar.

It was also observed that composition A is particularly effective in the treatment of SARS-CoV-2-induced peripheral neuropathic pain.

### Example 2

Another pharmaceutical composition in aqueous gel form according to the invention (composition A′) was prepared from the ingredients indicated in the table hereinafter, the quantities of which are expressed as a % by weight.

TABLE 3

| COMPOSITION A′ (Invention) | Quantity |
|---|---|
| Amitriptyline hydrochloride | 15 |
| Hydroxyethylcellulose | 1 |
| Propylene glycol | 5 |
| Methyl paraben | 0.1 |
| pH agent | Q.s. pH 5.5 ± 0.5 |
| Water | Q.s. 100 |

It was observed that the aqueous gel A′ according to the invention has a satisfactory skin penetration profile of amitriptyline and amitriptyline bioavailability.

It was also observed that composition A′ is particularly effective in the treatment of SARS-CoV-2-induced peripheral neuropathic pain.

### Example 3

Stability study of a formulation C in aqueous gel form (invention) and of a formulation B in cream form (comparative).

The following aqueous gel (composition C) was prepared from the ingredients indicated in the table hereinafter, the quantities of which are expressed as a % by weight.

TABLE 4

| COMPOSITION C (Invention) | Quantity |
|---|---|
| Amitriptyline hydrochloride | 10 |
| Hydroxyethylcellulose | 1 |
| Propylene glycol | 5 |
| PEG-7 Glyceryl cocoate | 2 |
| pH agent (NaOH, 1N solution) | Q.s. pH 5.5 ± 0.5 |
| Water | Q.s. 100 |

Composition B (cream) comprises 10% by weight of amitriptyline hydrochloride and 90% by weight of Excipial Hydrocreme® cream, marketed by Galderma, with respect to the total weight of composition B.

Each of compositions B and C was placed in an oven at a temperature of 40° C.

The stability of the compositions was then assessed visually over time (at T₀, when entering the oven; at T₂₄ₕ, 24 hours after entering the oven; and at T₃ₘₒₙₜₕₛ , 3 months after entering the oven).

The results of these extraction are compiled in the table below.

TABLE 5

| | Appearance | | |
|---|---|---|---|
| Compositions | at T₀ | at T_{24 h} | at T_{3 months} |
| Composition B | Opaque white | Phase separation | ND |
| (Comparative) | oil-in-water | observed. | |
| | emulsion | The top oily phase is | |
| | | white and opaque. The | |
| | | bottom aqueous phase is | |
| | | transparent | |
| Composition C | Translucent | Translucent | Translucent |
| (Invention) | colorless gel | gel | gel |

No syneresis was observed for composition C in aqueous gel form according to the invention after 3 months at 40° C.

A phase separation of comparative composition B in oil-in-water emulsion form is also observed after merely 24 hours at 40° C.

Furthermore, a full stability study was performed on composition C according to the invention for 6 months at 40° C.

The results are compiled in Tables 6 and 7 hereinafter.

TABLE 6

| | | RESULTS | | | | | | | |
|---|---|---|---|---|---|---|---|---|---|
| TESTS | SPECIFICATIONS | T0 | | T1 month | | T3 months | | T6 months | |
| Visual | Translucent | Translucent | | Translucent | | Translucent | | Translucent | |
| appearance | colorless gel | colorless gel | | colorless gel | | colorless gel | | colorless gel | |
| pH | 4.5 to 6.0 | 5.5 | | 5.6 | | 5.5 | | 5.3 | |
| Viscosity | 450 to | 1039 | mPa · s | 883 | mPa · s | 765 | mPa · s | 756 | mPa · s |
| | 1500 mPa · s | | | | | | | | |
| Amitriptyline | 95.0 mg/g to | 102.7 | mg/g | 99.9 | mg/g | 99.6 | mg/g | 100.6 | mg/g |
| HCl assay | 105.0 mg/g | | | | | | | | |

TABLE 7

| Amitriptyline HCl | | RESULTS | | | |
|---|---|---|---|---|---|
| degradation products | SPECIFICATIONS | T0 | T1 month | T3 months | T6 months |
| Cyclobenzaprine | ≤0.1% | <limit of detection | <limit of detection | <limit of detection | <limit of detection |
| Dibenzosuberone | ≤0.1% | Not detected | Not detected | Not detected | Not detected |
| Unknown impurities | Reported if ≤0.1%, | Not detected | Not detected | Not detected | Unknown impurity 1 |
| | None < 0.2% | | | | RRT 0.38 min < 0.1%; |
| | | | | | Unknown impurity 2 |
| | | | | | RRT 0.45 min < 0.1% |
| Total Impurities | ≤1% | N/A | N/A | N/A | <0.1% |

No syneresis was observed for composition C in aqueous gel form according to the invention after 6 months at 40° C.

No major variations were also observed on each of the tests conducted.

The good physicochemical stability of the compositions in aqueous gel form according to the invention can thus be noted.

It was also observed that composition C in aqueous gel form according to the invention is particularly effective in the treatment of SARS-CoV-2-induced peripheral neuropathic pain.

1. Method for treating coronavirus-induced neuropathic pain comprising the topical application of a pharmaceutical composition in aqueous gel form containing from 10 to 30% by weight, with respect to the total weight of the composition, of amitriptyline and/or one of the pharmaceutically acceptable salts thereof.

2. Method according to claim 1, for the treatment of coronavirus-induced peripheral neuropathic pain.

3. Method according to claim 1, for the treatment of SARS-CoV-2-virus-induced neuropathic pain.

4. Method according to claim 1, characterized in that the total content of amitriptyline and/or of one of the pharmaceutically acceptable salts thereof is between 10 and 25% by weight, with respect to the total weight of the pharmaceutical composition.

5. Method according to claim 1, characterized in that the total content of amitriptyline and/or of one of the pharmaceutically acceptable salts thereof is between 10 and 20% by weight, more preferably between 10 and 15% by weight, with respect to the total weight of the pharmaceutical composition.

6. Method according to claim 1, characterized in that the pharmaceutical composition further comprises at least one cellulose polymer; preferably chosen from cellulose ethers; more preferably from non-ionic cellulose ethers; even more preferably from (a) (C₁-C₄)alkylcelluloses such as methylcelluloses and ethylcelluloses, (b) (poly)hydroxy(C₁-C₄)alkylcelluloses such as hydroxymethylcelluloses, hydroxyethylcelluloses and hydroxypropylcelluloses, (c) mixed (poly)hydroxy (C₁-C₄)alkyl-(C₁-C₄)alkylcellulose celluloses such as hydroxypropyl-methylcelluloses, hydroxyethyl-methylcelluloses, hydroxypropyl-ethylcelluloses, hydroxyethyl-ethylcelluloses and hydroxybutyl-methylcelluloses, and (d) mixtures thereof more preferably, the composition comprises at least one (poly)hydroxy(C₁-C₄)alkylcellulose such as hydroxymethylcelluloses, hydroxyethylcelluloses and hydroxypropylcelluloses; and even more preferably, the composition comprises at least hydroxyethylcellulose.

7. Method according to claim 1, characterized in that the pharmaceutical composition further comprises at least one C₂-C₈ polyol; preferably chosen from C₃-C₆ polyol, ethylene glycol, and mixtures thereof; more preferably from propylene glycol, 1,3-propanediol, 1,3-butylene glycol, pentane-1,2-diol, dipropylene glycol, hexylene glycol, pentylene glycol, glycerol, ethylene glycol, and a mixture of these compounds; more preferably, the composition comprises at least propylene glycol.

8. Method according to claim 1, characterized in that the viscosity of the pharmaceutical composition, at a temperature of 20° C. and at atmospheric pressure, is between 400 and 2500 mPa·s; preferably between 600 and 2000 mPa·s; and more preferably between 800 and 1500 mPa·s.

9. Method according to claim 1, characterized in that the water content in the pharmaceutical composition is greater than or equal to 65% by weight, preferably between 65 and 90% by weight; more preferably between 70 and 90% by weight, even more preferably between 75 and 85% by weight, with respect to the total weight of the pharmaceutical composition.

10. Method according to claim 1, characterized in that the pharmaceutical composition is free from fatty substances.

11. Method according to claim 1, characterized in that the pharmaceutical composition is applied by the cutaneous route.

12. Method according to claim 1, characterized in that the pharmaceutical composition comprises:
(i) from 10 to 30% by weight of amitriptyline or one of the pharmaceutically acceptable salts thereof, preferably from 10 to 20% by weight, more preferably from 10 to 15% by weight, with respect to the total weight of the composition;
(ii) from 0.1 to 10% by weight of at least one cellulose polymer, with respect to the total weight of the composition;
(iii) from 0.1 to 15% by weight of at least one C₂-C₈ polyol, with respect to the total weight of the composition; and
(iv) water, preferably at a content ranging from 65 to 90% by weight with respect to the total weight of the composition.
